(19) United States
(12) Patent Application Publication
(10) Pub. No.: US 2008/0188818 A1
(43) Pub. Date: Aug. 7, 2008

(54) Method for predicting postoperative arterial bleeding following open heart surgery

(75) Inventor: Akio Wakabayashi, Murietta, CA (US)

Correspondence Address: WILLIE KRAWITZ, 3001 CHAPEL HILL ROAD, ORANGE, CA, 92867-1927, US

(73) Assignee: AKIO WAKABAYASHI RESEARCH FOUNDATION

(21) Appl. No.: 11/701,934

(22) Filed: Feb. 2, 2007

(51) Int. Cl. A61M 5/00

(52) U.S. Cl. 604/264

(57) ABSTRACT

Prediction of continued or excessive arterial bleeding following open heart surgery is facilitated by employing a high vacuum drainage of the chest cavity using a multi-perforated drainage tube. Bleeding rates before the chest is closed (I.e.,=open chest bleeding rates) are compared with bleeding rates after the chest is closed air-tight (I.e.,=closed chest bleeding rates) while a patient is still on the operating table. If the closed chest bleeding rates are higher or comparable to the open chest bleeding rates, it is likely that the patient is suffering from arterial bleeding. On the other hand, if the patient's closed chest bleeding rate is less than the open chest bleeding rate, it is probably safe to transfer the patient to the intensive care unit.

### BACKGROUND OF THE INVENTION

This invention relates to a new and improved method for predicting whether a patient is susceptible to continued, excessive bleeding following an open heart surgical procedure. This type of bleeding would necessitate returning the patient from an intensive care unit to the operating room for explorative examination, versus a normal recovery where excessive bleeding does not constitute a problem.

One serious complication following open heart surgery is excessive postoperative bleeding which necessitates returning a patient to the operating room for re-examination in an effort to determine and to control the bleeding. According to the database of the SOCIETY of THORACIC SURGEONS, 2002, the the incidence of necessitating a patient returning to the operating room was 2.63%, of 200,935 first time open heart surgery patients. In the case of second time (redo) heart surgery, the incidence of requiring a patient returning to the operating room was 3.98%, of 20,775 patients.

References concerning postoperative bleeding include:
SOCIETY OF THORACIC SURGEONS, “CARDIAC SURGERY DATABASE 2002”;
CARDIAC SURGERY, KIRKLIN J W, ET AL New York, Wiley 2d ed. 1993: 222-24;
ANN. THORAC SURGERY, 1995, 59: 664-7 “Resteronotomy for Bleeding After Cardiac Operations: A Marker for Increased Morbidity and Mortality”, Unsworth—White, et al;
ARCH. SURG. 1998, 133:442-447, “Reexploration for Hemorrhage Following Coronary Artery Bypass Grafting”, Dacey L J, et al;
J. THORAC CARDIOVASCULAR SURGERY, 1996, 111:1037-46, “Reexploration for Bleeding is a Risk Factor For Adverse outcomes After Cardiac operations”, MOULTON, M J, et al;
ASAIO J. 2003: 49 (May-June) 300-303, “High vacuum Drainage of the Chest 8. Using a Miniature Double Lumen Chest Tube”, WAKABAYASHI, A. and, ROOHK, V.;
ASAIO J. 2006, 52 (March-April) 215-216, “Clinical Evaluation of the Safety of High Vacuum Chest Drainage”, WAKABAYASHI, A.; and,
U.S. Pat. No. 6,299,593—WAKABAYASHI, A.

Patient bleeding rates following open heart surgery are evaluated during about a five hour period in the intensive care unit to determine which patient is suffering from excessive bleeding to the point where a return to the operating room becomes necessary. Exceptions are of course massive bleeding or cardiac tamponade. However, once a patient has been transferred to the intensive care unit and continues to bleed, it is difficult for a surgeon to decide if a return to the operating room is necessary.

However, if a method were made available to predict which patient required take-back to the operating room, for excessive postoperative bleeding, before the patient leaves the operating room after an open heart surgical procedure, significantly higher morbidity and mortality rates and hence longer hospital stay and cost could be reduced. Problems associated in dealing with excessive postoperative bleeding are discussed, supra, in the reference papers authored by Unsworth—White, et al; Dacey, L J, et al; and, Moulton M J, et al.

It is now known that high vacuum drainage is not as dangerous as previously thought (for over 50 Years) due to suction injury to soft tissue such as lung tissue unless high vacuum generates excessive suction force against these tissues. Since suction force is a product of vacuum pressure multiplied by the opening size of a drain tube, it can be controlled by making the opening size diameter substantially smaller. This is disclosed in the inventor's U.S. Pat. No. 6,299,593 and incorporated herein by reference. An FDA exemption for such a device was obtained Aug. 23, 2002 as Rc: CO20022, Regulatory Class: Class II exempt. The trade name of such types of Applicant's tubes is “NO-NUMO”™. It was found that when perforated tubes, preferably of the type described, supra, are inserted into the mediastinum following routine open heart surgery, and high vacuum pressure is applied, the bleeding rate was substantially less, and postoperative bleeding ceased more quickly than a conventional low pressure vacuum system.

This beneficial effect is known as internal compression hemostasis. Hence, when all the air inside the mediastinum is aspirated quickly by high vacuum pressure, soft tissue such as pericardium and adipose tissue are firmly sucked against the heart and blood vessels, thereby inducing internal compression hemostatis; this will reduce bleeding from non-arterial bleeding sources.

### THE INVENTION

Subsequent to these developments by the inventor herein, It has now been discovered that it could be possible to predict postoperative arterial bleeding while the patient is still on the operating table. It is known that high vacuum, such as 40 pKa, can be safely be applied to multi-perforated tubes with the largest round opening of 2 mm in diameter for chest drainage. When these tubes are placed inside the mediastinum during the closing procedure, the tubes continue to aspirate blood, in contrast to a conventional low vacuum drainage system that does not aspirate blood until the mediastinum is completely closed. During the closing procedure, vacuum pressure of the vacuum pump remains below a preset value of vacuum pressure, e.g., 40 pKa. until the mediastinum is completely closed, because the vacuum pump is still suctioning atmospheric air. At the moment when the mediastinum is closed air-tight, the vacuum pressure then jumps to the preset level. The bleeding rate before the vacuum pressure gauge reaches the set-value is defined as the “Open Chest Bleeding Rate”, and the bleeding rate after the vacuum pressure reaches the set-value is defined as the “Closed Chest Bleeding Rate”. Bleeding rates are calculated based on volume divided by time. It is believed the set point may vary from about 10 pKa to 55 Pka, and 40 pKa is considered suitable.

Basically, inducing internal compression hemostatis following open heart surgery by the method described in Applicant's U.S. Pat. No. 6,299,593 results in soft tissue such as the heart sac or fat tissue surrounding the heart to be firmly suctioned against the heart. As a result, bleeding rates substantially diminish after the chest is completely closed. However, while internal compression hemostasis is obviously desirable, it is not strong enough to control arterial bleeding.

Hence, according to the invention, it has been found that for patients making an uneventful recovery, the closed-chest bleeding rate was significantly less than the open-chest bleeding rate, and this finding was observed in forty-six patients. By comparison, two patients who suffered bleeding following transfer to the intensive care unit needed to be returned to the operating room for re-exploration of the mediastinum, since they had exhibited closed-chest bleeding rates which remained unchanged or higher than their open-chest bleeding rates. Both patients were found to have arterial bleeding which could not be controlled by internal compression hemostasis, thereby necessitating their return to the operating room.

Generally speaking, if the closed-chest bleeding rate decreases within about twenty-thirty minutes, the surgeon will continue to close the patient's chest and transfer the patient to the intensive care unit. However, if the closed-chest bleeding rate does not come down within this time frame, it may be necessary for the surgeon to continue monitoring the patient to determine if re-exploration is appropriate.

It is believed that an observation period of say a further thirty to sixty minutes would likely provide an adequate time frame for concluding re-exploration of the mediastinum would be necessary or whether a transfer to the intensive care unit would be appropriate. This would permit both the hospital and the surgeon sufficient time in which to assess the patient's condition and to reduce the risk to the patient and cost to the hospital.

It will be appreciated that the determination of open and closed chest bleeding rates in conjunction with the inventor's high vacuum suction pressure to induce internal compression hemostatis as disclosed in his prior U.S. Pat. No. 6,299,593 has not previously been known.

1. A method for determining excessive bleeding rates from the mediastinum of a patient following open heart surgery comprising the steps of applying high suction pressure to the mediastinum through a multi-perforated suction tube to induce internal compression hemostatis, calculating the bleeding rate of the patient before complete chest closure to determine the open chest bleeding rate based on a set value of vacuum pressure while the suction device is still suctioning air, determining the closed chest bleeding rate following chest closure based on the set value, and observing the patient for a period of time to determine if the closed chest bleeding rate decreases, thereby justifying transferring the patient from an operating room setting to an intensive care unit.

2. The method of claim 1, in which the set value is about 10 pKa to 55 pKa.

3. The method of claim 1, in which the period for observation of the patient following chest closure varies from about 20-30 minutes.

4. The method of claim 3, in which the period for observation of the patient varies from about a further 30-60 minutes.

5. The method of claim 1, in which a perforation size of the suction tube is not greater than about 2 mm in diameter, at 40 pKa.

6. The method of claim 1, in which the set value is about 40 pKa.
